(19) United States
(12) Patent Application Publication
(10) Pub. No.: US 2019/0116808 A1
(43) Pub. Date: Apr. 25, 2019

(54) CANNABINOID DELIVERY VIA CONSUMABLE FOODSTUFFS

(71) Applicants: Joseph A. Tedeschi, Manhattan Beach, CA (US); James DeSisto, El Segundo, CA (US)

(72) Inventors: Joseph A. Tedeschi, Manhattan Beach, CA (US); James DeSisto, El Segundo, CA (US)

(21) Appl. No.: 15/792,657

(22) Filed: Oct. 24, 2017

(51) Int. Cl. A21D 2/14; A21D 10/02; A21D 13/00; A61K 36/185; C07C 39/23; C07D 311/78

(52) U.S. Cl. CPC A21D 2/14; A21D 10/025; C07D 311/78; A61K 36/185; C07C 39/23; A21D 13/00

(57) ABSTRACT

Methods and apparatuses for improving the conversion of cannabolic acids to active pharmacological cannabinoids in various foodstuffs are disclosed.

### BACKGROUND

### Field

Aspects of the present disclosure generally relate to medicinal delivery of cannabinoids, and more specifically to improved delivery methods for cannabinoid delivery via consumable foodstuffs.

### Background

Recently, certain biomass materials have become legalized in certain jurisdictions. The plant family Cannabaceae, including the species Cannabis, having three species, i.e., cannabis sativa, cannabis indica, and cannabis ruderalis, has been legalized for personal use in several state jurisdictions. In Colorado, for example, approximately 700 million dollars' worth of Cannabis was sold during 2014.

Cannabis is grown and harvested as a multi-component field crop. The plant can be divided into fiber, seeds and flowers. Each of these parts have different potential uses in commerce, and, as such, each part and/or combination of parts may undergo different chemical and/or mechanical processing techniques in order to extract, separate and refine various constituents of the plant. In addition the cannabis plants, both the indica and sativa varieties, offer functionality for chemical engineering operations not yet available from traditional materials.

Because of the newly acquired legal status for certain uses of cannabis, growers and farmers have begun hybridizing cannabis plants to increase and/or alter the amount of the active ingredients in cannabis plants. One of the more well-known active ingredients is known as Delta-9 tetrahydrocannabinol, more commonly referred to as “THC.”

The hybridization of various strains of cannabis, however, may be done to produce various levels of cannabinoids and/or other chemical compounds, e.g., Cannabinol (CBN), Delta-8 tetrahydrocannabinol, Cannabinodiol (CBD), Cannabichromene (CBC), Delta-9 tetrahydrocannabinol, Cannabidiolic acid (CBDA), Cannabidivarin (CBDV), Cannabidivarinic acid (CBDVA), Cannabicyclol (CBL), Cannabinol methylether (CBNM), terpinoids, fatty acids, flavonoids, phenols, etc. A list of some of the cannabinoids and/or other chemical compounds that may be present in a given strain of cannabis may be found in Forensic Science and Medicine: Marijuana and the Cannabinoids, M. A. ElSohly, Ed., Humana Press Inc., Totowa, N.J., which is expressly incorporated by reference herein. Each cannabinoid may provide antiemetic, euphoric, anti-inflammatory, analgesic, and/or antioxidant pharmacological effects to a person that ingests a substance containing that specific cannabinoid. Further, combinations of cannabinoids may have other and/or additional pharmacological effects.

To change various cannabinoid and/or other chemical compound levels in a cannabis plant, a strain of cannabis sativa may be hybridized with a strain of cannabis indicia. Although this hybridization may control a specific cannabinoid and/or chemical compound level, other cannabinoids that contribute to the overall pharmacological effect of the hybridized plant may be removed and/or reduced, thereby rendering the hybridized plant less suitable than desired.

Other approaches have been undertaken to extract specific cannabinoids and/or other chemical compounds from biomass solids. The cannabis plants are directly exposed to solvents, such as butane, and the oils and resins e.g., butane honey oil (BHO), shatter, etc., from the cannabis plants are extracted. With such approaches, however, at least some of the solvent remains in the final product, and is thus consumed along with the cannabinoids. The pharmacological effects of the solvents may add to those of the cannabinoids, however, the pharmacological effects of the solvents are often harmful to humans. Further, the open exposure of plants to butane or other flammable solvents may be done in uncontrolled environments, and as such poses extreme safety risks for extraction of cannabinoids in such a manner.

Medicinal and/or recreational administration of cannabis may also be through consumption by a patient. For example, cannabis may be placed in capsules, etc., for absorption of the cannabinoids through ingestion.

### SUMMARY

A method of delivering cannabinoids in accordance with an aspect of the present disclosure includes pairing a dough product with a cannabinoid-containing material to create a mixed dough product. The pairing is based at least in part on cooking, heating, and/or re-heating conditions which the mixed dough product will be subjected to. The mixed dough product is then shaped and packaged.

In various aspects of the present disclosure, the method further optionally includes the pairing being at least partially based on at least one specific cannabinoid present in the cannabinoid-containing material, at least partially cooking the mixed dough product prior to packaging, and the cooking, heating, and/or re-heating conditions being modified to change an amount of carboxylation of a cannabinoid in the cannabinoid-containing material.

A cannabinoid-containing food product in accordance with an aspect of the present disclosure includes a dough-based product, and a cannabinoid-containing material. The cannabinoid-containing material is paired with the dough-based product to form a mixed dough product. The pairing of the cannabinoid-containing material and the dough-based product is based at least in part on cooking, heating, and/or re-heating conditions which the mixed dough product will be subjected to.

In various aspects of the present disclosure, the cannabinoid-containing food product may also include the pairing being based at least in part on at least one specific cannabinoid present in the cannabinoid-containing material, the mixed dough product being at least partially cooked prior to delivery to a consumer, and the cooking, heating, and/or re-heating conditions of the mixed dough product being modified to change an amount of carboxylation of a cannabinoid in the cannabinoid-containing material.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further purposes and advantages, will be better understood from the following description when considered in connection with the accompanying figures. It is to be expressly understood, however, that each of the figures is provided for the purposes of illustration and description only and is not intended as a definition of the limits of the present disclosure.

### BRIEF DESCRIPTION OF THE DRAWINGS

For a more complete understanding of the present disclosure, reference is now made to the following description taken in conjunction with the accompanying drawings.

FIG. 1 illustrates a process flow in accordance with an aspect of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts. As described herein, the use of the term “and/or” is intended to represent an “inclusive OR”, and the use of the term “or” is intended to represent an “exclusive OR”.

### Overview

Because cannabis has been recently legalized in many jurisdictions, there is a renewed interest in medicinal and other uses of cannabis. For example, and not by way of limitation, cannabis is a unique source for a family of functionally active chemicals called cannabinoids. The extent of the specific cannabinoids is recently emerging as a new set of chemical feedstocks, which may be used to produce many functional products. Cannabis plants also yield fibers and/or other biomass material that can be converted into textiles, nonwovens, and traditional pulp based stock, biofuels and/or bulk solid materials. These refined cannabis fibers can replace and/or improve synthetically derived fibers that are prevalent in many applications.

Although recreational use of cannabis, which remains illegal under federal law, focuses on THC content of a given strain of cannabis, cannabis may also be grown and/or hybridized to focus the plant yield on other cannabinoids, cannabis fiber, flowers, and/or seeds. These hybridized and/or highlighted qualities of the cannabis plant can increase the yield of specific cannabinoids from certain varieties. For example, and not by way of limitation, a cannabis strain that is focused on seed production may be unsuitable for other cannabis uses, but may increase the yield for product conversion feedstocks.

In an aspect of the present disclosure, the cannabis strain may be optimized to produce a specific cannabinoid when exposed to a certain environment. For example, and not by way of limitation, a cannabis strain may be hybridized to produce higher levels of a desired cannabinoid when exposed to a certain temperature for a specified amount of time. In such an aspect of the present disclosure, a specific strain may be used for providing higher relative concentrations of a desired cannabinoid when baked in pizza or cookie dough, while a different strain may be used for providing higher relative concentrations of a desired cannabinoid when consumed in pill or capsule form. Further, combinations of strains and/or extracted oils may be applied in a given application to provide a more standardized pharmacological effect.

In an aspect of the present disclosure, the control of certain parameters may increase the yield of desired end-product constituents. Such parameters include, but are not limited to: 1) moisture, i.e., the weight percentage of water in the total mass of the plant; 2) particle size; i.e., the maximum dimension of the particle measured by the collection of particles as they pass through a series of calibrated sized screens; 3) processing temperature; 4) processing pressure; 5) concentrations, e.g., measurements that relate to the mass ratio of one constituent to another or the individual constituent to the total mass in the specific sample at a certain point in the process flow; 5) viscosity, i.e., the physical separation and purification of the various constituents depends upon the flow properties of the material, which also takes into account the relationship between viscosity and shear rate (rheology) at the point in the process where viscosity is measured; and 7) pH, O2, and/or other analytically derived specific constituent measurements, where processing of each measured constituent, e.g., pH, may be affected by the ratios of each of these measurable parameters in combination with the concentration of the target constituents.

To control the various parameters listed above, and/or other processing parameters to extract the desired end-product (“desired constituents”), an aspect of the present disclosure may control not only the parameters, but the order in which the parameters are controlled.

In an aspect of the present disclosure, dough products, e.g., pizza crusts, wraps, bread to be toasted, etc., and/or other foodstuffs that may be re-heated, may be employed as delivery mechanisms for cannabinoids. When re-heatable foodstuffs are used as such a mechanism, the re-heating process may increase the active cannabinoid level within the foodstuff.

Many cannabinoids are present in cannabis strains as acids, e.g., Delta-9 tetrahydrocannabinolic acid (THCA), Cannabidiolic acid (CBDA), etc. Pharmacological cannabinoids are produced by decarboxylating these acids to active cannabinoids. Decarboxylation is a chemical reaction that removes a carbon atom from a carbon chain, which reduces the acid to an active component while releasing carbon dioxide (CO2). Depending on the temperature, pressure, and other characteristics present during this conversion, certain cannabinoid acids can be converted into their corresponding active components, rather than being converted into other cannabinoids that may have fewer pharmacological effects. For example, and not by way of limitation, CBDA may be converted into CBD under the proper conditions, but under other conditions may be converted to CBN, which has fewer pharmacological effects than CBD.

In an aspect of the present disclosure, the re-heating of foodstuffs (e.g., pizza dough, bread, etc.) may allow for a lower amount of active cannabinoids to be present in the original dough; however, through re-heating and/or additional heating of the foodstuff a sufficient amount of the desired active cannabinoid will be present in the foodstuff In another aspect of the present disclosure, the re-heating of foodstuffs may allow for an increase in the amount of desired active cannabinoid present in the foodstuff which may not be possible without the re-heating and conversion of cannabinoid acids during the re-heating process.

Because each of the cannabinoid acids decarboxylases at a different temperature, pressure, pH, etc., in an aspect of the present disclosure, a specific type of cannabinoid acid is selected to be added to a particular foodstuff, with a priori (prior) knowledge of how the foodstuff will be prepared, such that the desired active cannabinoid is relatively increased in the re-heated product. For example, and not by way of limitation, the instructions to cook a frozen pizza may be indicated as a 25 minute cooking time at 400° F. If such a foodstuff is sold in the United States, the average atmospheric pressure is between 11 pounds per square inch and 15 pounds per square inch. As such, the temperature and pressure conditions for cooking are known prior to the actual heating for that particular foodstuff. Insertion or inclusion of appropriate cannabinoid acids in the pizza dough and/or other pizza ingredients allows for the relative amounts of specific active cannabinoids to be increased without significantly increasing any unwanted active cannabinoids in the heated and/or re-heated food product. Other characteristics, e.g., pH, may also be known and/or supplied to the heating process to increase the carboxylation of the cannabinoid acids into desired active cannabinoids, while reducing the degradation of the cannabinoid acids into undesired byproducts. Other temperatures, times, pH conditions, and/or pressure conditions may be employed without departing from the scope of the present disclosure.

Further, the heating and/or re-heating process may be defined differently for cannabinoid acid-infused foodstuffs to provide similar cooking conditions for the foodstuff; however the changed conditions may provide additional conversion of the cannabinoid acids into desired active cannabinoids. For example, and not by way of limitation, rather than cooking at 400° F. for 25 minutes, the cannabinoid-infused pizza may be cooked at 300° F. for 30 minutes. The lower temperature for longer period may cook the dough similarly, but may affect the conversion of cannabinoid acid to active cannabinoid rate in a different fashion. Lower temperatures may only affect some of the cannabinoid acids, while leaving other cannabinoid acids unchanged, thus further providing control over the cannabinoid acid conversion process. Further, other cannabis constituents such as terpenes (i.e., the oils that give plants and flowers their unique smells such as berry, mint, citrus, pine, etc.), may remain in greater concentrations when temperatures for heating and/or re-heating are reduced. Terpenes provide additional pharmacological benefits, e.g., stress relief, increased focus and/or awareness, etc., just as botanical oils do.

### Foodstuff Cooking Parameters

In an aspect of the present disclosure, cannabinoid containing material may be added to various foodstuffs that will be heated and/or re-heated. In such an aspect, the temperature, time, and/or other cooking conditions may be taken into account when adding the cannabinoid containing material. For example, and not by way of limitation, pizza is considered fully cooked when the pizza dough reaches an interior temperature of between 200-205° F., but not more than 210° F. In an aspect of the present disclosure, cannabinoid containing material that converts cannabinoid acids to desired active cannabinoids may be added to the pizza dough and/or placed on the pizza dough. The cannabinoid containing material added to the pizza dough may be selected to have conversion (e.g., carboxylation) of the cannabinoid containing material at that temperature range.

Although described with respect to specific strains of cannabis, the present disclosure also includes the use other cannabinoid materials, e.g., BHO, etc., that may have desirable carboxyl conversion conditions for given heating/re-heating conditions. Related art approaches do not appreciate or consider the conditions that a particular cannabinoid would be created, and, as such, the present disclosure provides advantages over such previous efforts in this arena.

For example, and not by way of limitation, the related art does not specify which types of cannabinoids are best carboxylated in certain cooking conditions, as there is no one “optimal” way to convert a cannabinoid acid to the desired active cannabinoid. The related art does not take into account the different ingestion rates for liquids and solids, and thus the related art does not properly determine the rate of cannabinoid ingestion for various cannabinoid products. In an aspect of the present disclosure, certain cannabinoids can have a controlled ingestion rate by controlling the amount of the desired cannabinoid in the foodstuff via the heating/re-heating process. In another aspect of the present disclosure, oils and liquids heat up more slowly as compared to dough products, e.g., pizza crusts that are inherently filled with air bubbles. As such, during the heating process, oils and liquids would fall short of increasing desired cannabinoid levels whereas pizza crusts will relatively increase desired cannabinoid levels for the same amount of cannabinoid acid.

In an aspect of the present disclosure, heated and/or re-heated dough products such as pizza crust may be more readily suited for cannabinoid delivery as a consumable food product, because dough products provide more desirable conditions for the conversion (e.g., carboxylation) of “raw” cannabinoid acids to desired active cannabinoids. Such dough products infused with cannabinoid containing material may also add to the flavor of the dough products because the terpenes are preserved in the temperature/time/cooking conditions for dough products when the cannabinoid containing material is properly selected. Such selection may also increase the therapeutic value of the consumable food product as opposed to attempts to increase the amount of cannabinoid containing material or delivering the cannabinoids via liquid ingestion. The pairing of particular types of cannabinoid containing material (e.g., specific strains of cannabis, cannabis oils, etc.) to deliver desired cannabinoids in a particular food product provides synergistic advantages not recognized or understood by the related art.

In some aspects, incorporating cannabinoid containing material may also provide other advantages to the food product. For example, and not by way of limitation, cannabinoid containing material, once infused into dough products, may produce a slightly acidic dough product. The slight acidity of the product may increase the shelf life of the product beyond the shelf life of the same product without the cannabinoid containing material infusion. The cannabinoid containing material may act as a natural preservative and may also protect food quality by limiting starch degradation.

Popular foods, such as pizza, toasted sandwiches, etc., may also provide a socially and/or morally acceptable method for therapeutic delivery of the desired cannabinoids. For example, and not by way of limitation, annual pizza sales revenue in the United States alone is $32 billion, and the total number of pizzerias in the United States is 70,000. Because it is not often socially and/or morally wrong to consume pizza, the inclusion of cannabinoids in pizza crust for medicinal purposes may avoid the stigma of ingestion of cannabinoids in some other manner.

Because the carboxylation process produces CO2, the addition of cannabinoid containing material into dough products may also produce better heated and/or re-heated food products even if there are no medicinal or therapeutic benefits. For example, dough products may trap some of the additional CO2 that is produced, giving the baked dough product more “air” and/or lightness, in addition to additional flavors infused in the final baked product. Such qualities are desirable in baked goods. The crusts of such baked goods may also be healthier than non-cannabinoid infused goods, as cannabinoids may produce a higher glycemic index in the crust.

Other foodstuffs infused or otherwise including cannabinoid-containing material may be pre-baked or warmed prior to delivery to consumers. Such foodstuffs may thus include cannabinoid-containing material that has already been partially carboxylated, and additional carboxylation may be performed by the consumer.

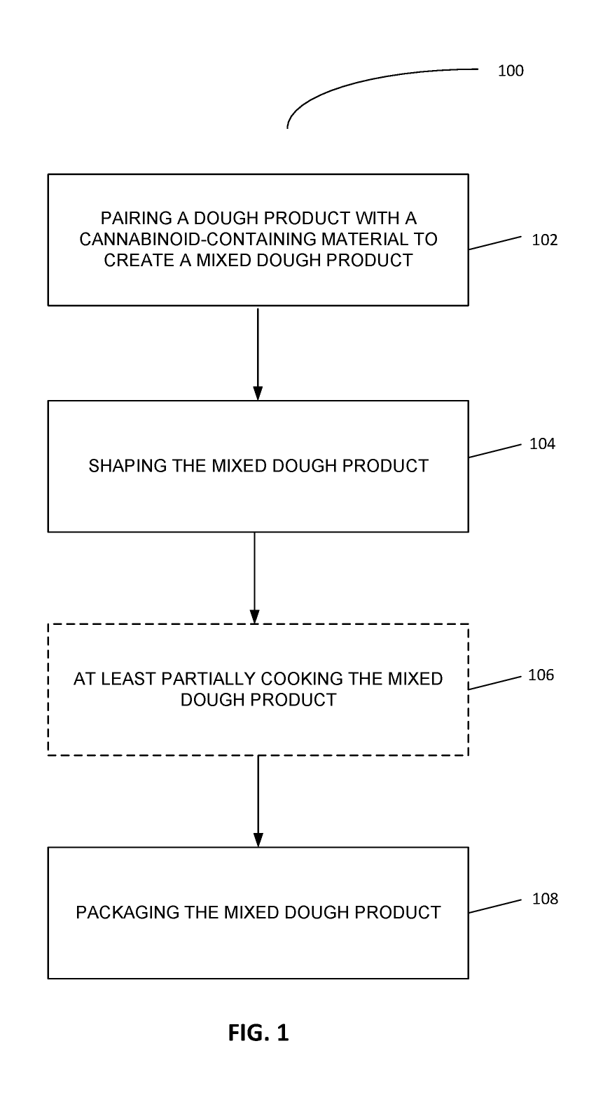

FIG. 1 illustrates a process flow in accordance with an aspect of the present disclosure.

Process 100 describes a method of pairing a dough product with a cannabinoid-containing material. Process 100 comprises block 102, which illustrates pairing combining a cannabinoid-containing material with a dough product to create a mixed dough product. The combination may be based at least in part on the conditions which the mixed dough product will be subjected to cooking, heating, and/or re-heating. The combination may also be based at least in part on at least one specific cannabinoid present in the cannabinoid-containing material.

Process 100 also comprises block 104, which illustrates shaping the mixed dough product. Process 100 also comprises optional block 106, which illustrates at least partially cooking the mixed dough product. Process 100 also comprises block 108, which illustrates packaging the mixed dough product.

Although the present disclosure and its advantages have been described in detail, it should be understood that various changes, substitutions and alterations can be made herein without departing from the technology of the disclosure as defined by the appended claims. For example, relational terms, such as “above” and “below” may be used with respect to a device. Of course, if the device is inverted, above becomes below, and vice versa. Additionally, if oriented sideways, above and below may refer to sides of a device. Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present disclosure. Accordingly, the appended claims are intended to include within their scope such processes, machines, manufacture, compositions of matter, means, methods, or steps.

The description of the disclosure is provided to enable any person reasonably skilled in the art to make or use the disclosure. Various modifications to the disclosure will be readily apparent to those reasonably skilled in the art, and the generic principles defined herein may be applied to other variations without departing from the spirit or scope of the disclosure. Thus, the disclosure is not intended to be limited to the examples and designs described herein, but is to be accorded the widest scope consistent with the principles and novel features disclosed herein.

Although several embodiments have been described in detail for purposes of illustration, various modifications may be made without departing from the scope and spirit of the present disclosure. Accordingly, the disclosure is not to be limited by the examples presented herein, but is envisioned as encompassing the scope described in the appended claims and the full range of equivalents of the appended claims.

1. A method of delivering cannabinoids, comprising:
pairing a dough product with a cannabinoid-containing material to create a mixed dough product, in which the pairing of the dough product with the cannabinoid-containing material is based at least in part on a cannabinoid present in the cannabinoid-containing material and cooking, heating, and/or re-heating conditions which the mixed dough product will be subjected to;
shaping the mixed dough product; and
packaging the mixed dough product.

2. The method of claim 1, in which the pairing of the dough product with the cannabinoid-containing material is based at least in part on at least one specific cannabinoid present in the cannabinoid-containing material.

3. The method of claim 1, further comprising at least partially cooking the mixed dough product prior to packaging.

4. The method of claim 1, in which the cooking, heating, and/or re-heating conditions are modified to change an amount of carboxylation of a cannabinoid in the cannabinoid-containing material.

5. A cannabinoid-containing food product, comprising:
a dough-based product; and
a cannabinoid-containing material, paired with the dough-based product to form a mixed dough product, in which the pairing of the cannabinoid-containing material and the dough-based product is based at least in part on a cannabinoid present in the cannabinoid-containing material and cooking, heating, and/or re-heating conditions which the mixed dough product will be subjected to.

6. The cannabinoid-containing food product of claim 5, in which the pairing of the dough product with the cannabinoid-containing material is based at least in part on at least one specific cannabinoid present in the cannabinoid-containing material.

7. The cannabinoid-containing food product of claim 5, in which the mixed dough product is at least partially cooked prior to delivery to a consumer.

8. The cannabinoid-containing food product of claim 5, in which the cooking, heating, and/or re-heating conditions of the mixed dough product are modified to change an amount of carboxylation of a cannabinoid in the cannabinoid-containing material.
